(19) United States
(12) Patent Application Publication
(10) Pub. No.: US 2006/0014973 A1
(43) Pub. Date: Jan. 19, 2006

(54) Processes for the preparation of 16beta-alkoxy, 17alpha-hydroxy steroids and steroidal 16beta, 17alpha-diols from 16alpha, 17alpha-epoxy steroids

(75) Inventors: Yanzhong Wu, Bronx, NY (US); Sreenivasulu Megati, New City, NY (US); Charles C. Wu, Denville, NJ (US); Jianxin Ren, Nanuet, NY (US); Panolil Raveendranath, Monroe, NY (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/182,557

(22) Filed: Jul. 15, 2005

Related U.S. Application Data
(60) Provisional application No. 60/589,110, filed on Jul. 19, 2004.

(51) Int. Cl. C07J 1/00

(52) U.S. Cl. 552/618

(57) ABSTRACT

The present invention provides processes for the preparation of 16β-alkoxy, 17α-hydroxy steroids via the reaction of a 16α,17α-epoxy steroid with an appropriate alcohol in the presence of base. The present invention also provides processes for the preparation of 16β-alkoxy, 17α-hydroxy steroids.

### CROSS REFERENCE

This application claims benefit of U.S. Provisional Application Ser. No. 60/589,110 filed Jul. 19, 2004, which is incorporated herein by reference in its entirety.

### FIELD OF THE INVENTION

The present invention relates to processes for the preparation of 16β-alkoxy, 17α-hydroxy steroids via the reaction of a 16α,17α-epoxy steroid with an appropriate alcohol in the presence of base. The 16β-alkoxy, 17α-hydroxy steroids thus formed may be further elaborated to 16β,17α-steroidal diols.

### BACKGROUND OF THE INVENTION

Estriols are steroidal compounds of Formula I, wherein the term estriol refers to the three hydroxy groups located on the core estratriene structure at the 3,16 and 17 positions. 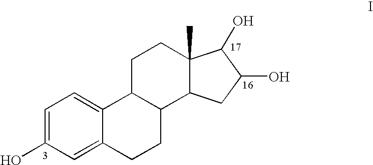
(3, 16, 17)-Estriols occur in four possible configurations: (16α,17α), (16α,17β), (16β,17α) and (16β,17β).

Estriols having the 16β,17a configuration are estrogenically-active components of the widely marketed pharmaceutical product Premarin®. Premarin® is used in estrogen and hormone replacement therapy regimens due to its proven ability to prevent various vasomoter disturbances (e.g., hot flushes, night sweating, etc.) and osteoporosis in peri- and post-menopausal women. The 16β,17α-estriol component can be present and active as both a free or sulfate conjugated phenol. Syntheses of 16β,17α estriols have been reported, however, the reported procedures are lengthy and involve separation to remove an unwanted isomer. See Fishman, J., Biggerstaff, W. R., J. Org. Chem., (1958),23:1190; Huffman, M. N., Lott, M. H., Tillotson, A. J., J. Biol. Chem., (1955), 217:107; Rhone, J. R., Huffman, M. N., Tetrahedron Lett., (1965), 1395-98. Accordingly, improved synthetic routes to 16β, 17α-estriols and key intermediates are needed.

### SUMMARY OF THE INVENTION

In some embodiments, the present invention provides processes for preparing a steroid having the c, d-ring structure of Formula I: 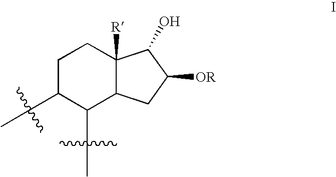
wherein:

R is C₁₋₆ alkyl or benzyl where the phenyl ring of the benzyl group is optionally substituted with from 1 to 3 substituents independently selected from the group consisting of C₁₋₃ alkyl, halogen, C₁₋₃ alkoxy, CO₂C₁₋₆ alkyl, C₁₋₆ thioalkyl, OH, cyano, nitro, N(C₁₋₃ alkyl)₂ and phenyl; and R′ is C₁₋₃ alkyl; comprising:

a) reacting a steroid having the c, d-ring structure of Formula II: 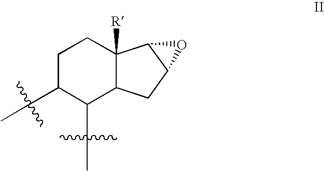
with a compound of formula HO—R in the presence of a base for a time and under conditions effective to form the compound of Formula I.

In some embodiments, R is benzyl and R′ is methyl. In some further embodiments, the base is a group I or II metal hydride or metal t-butoxide. In some embodiments, the base is sodium t-butoxide.

In some embodiments, the reaction is performed in a solvent, for example, an alcohol solvent. In some embodiments, the solvent is an alcohol of formula HO—R, for example, benzyl alcohol.

In some embodiments, the reaction of step (a) further comprises a cosolvent. In some embodiments, the cosolvent is 1-methyl-2-pyrrolidinone.

In some further embodiments, the reaction of step (a) comprises adding the steroid having the c, d-ring structure of Formula II and the 1-methyl-2-pyrrolidinone to a mixture of the compound of Formula R—OH and the base.

In still further embodiments, R is benzyl, R′ is methyl, the base is sodium t-butoxide, and the reaction of step (a) is performed in excess benzyl alcohol solvent and also, in the presence of a cosolvent that is 1-methyl-2-pyrrolidinone. In some such embodiments, the reaction of step (a) comprises:

(i) reacting the sodium t-butoxide with an excess of the benzyl alcohol to form a reaction mixture thereof; and

(ii) adding a mixture of the steroid having the c, d-ring structure of Formula II and the 1-methyl-2-pyrrolidinone to the reaction mixture of step (i).

In some embodiments, the processes of the invention further comprise the step of (b), which involves removing the group R from the steroid having the c, d-ring structure of Formula I to provide a steroid having the c, d-ring structure of Formula III:

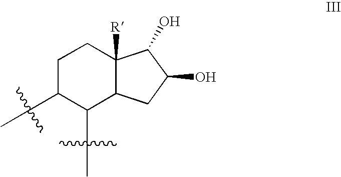

In some embodiments, the removing of step (b) is performed with hydrogen and a metal catalyst. In some such embodiments, the metal catalyst is Pd on carbon. In some such embodiments, the Pd is present from about 5 to about 10% by weight.

In some embodiments, the steroid having the c, d-ring structure of Formula II has the structure: 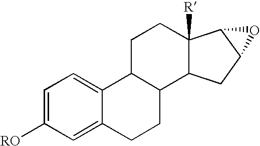
and the steroid having the c, d-ring structure of Formula I has the structure: 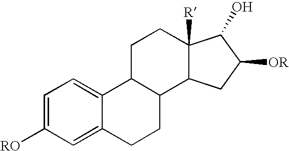
wherein each R is benzyl; and R′ is methyl.

In some embodiments, the steroid having the c, d-ring structure of Formula II has the structure: 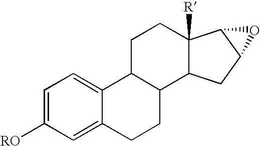
and the steroid having the c, d-ring structure of Formula I has the structure: 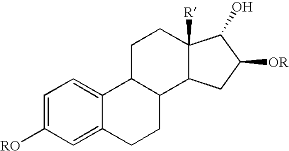
wherein each R is benzyl, and R′ is methyl, and the steroid having the c, d-ring structure of Formula III has the structure:

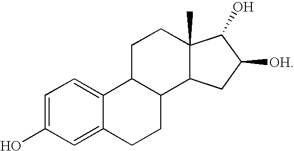

In some embodiments, the benzyl alcohol is heated to greater than about 40° C., or between about 50° C. and about 60° C., after being treated with sodium t-butoxide. In some embodiments, the benzyl alcohol is heated for between about 5 minutes and about 60 minutes after the addition of the sodium t-butoxide.

In some embodiments, the reaction mixture is heated after the addition of the steroid having the c, d-ring structure of Formula II, for example to a temperature greater than about 100° C., or between about 130° C. and about 150° C., after the addition of the steroid having the c, d-ring structure of Formula II. In some such embodiments, the reaction mixture is heated from about 5 to about 40 hours after the addition of the steroid having the c, d-ring structure of Formula II. In some embodiments, the reaction mixture is heated at about 140° C. for about 21 hours and at about 145° C. for about 7 hours after the addition of the steroid having the c, d-ring structure of Formula II.

In some embodiments, the product, e.g., the steroid having the c, d-ring structure of Formula I, is collected by precipitation, for example, by cooling the reaction mixture and adding methanol and water. In some such embodiments, the ratio of the initial volume of benzyl alcohol, to the volume of methanol added, to the volume of water added, is respectively, about 0.4 to about 1 to about 0.8. In some further embodiments, the precipitated product having the c, d-ring structure of Formula I is isolated and washed with a mixture of an alcohol and water, for example, methanol and water. In some embodiments, the methanol to water ratio in the wash is about 1 volume to about 4 volumes, respectively.

The present invention further provides products of the processes disclosed herein.

The present invention further provides compounds having the Formula I: 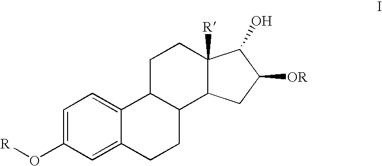
wherein:

R′ is C₁₋₃ alkyl; and

each R is an independently selected benzyl group where the phenyl ring of each benzyl group is optionally substituted with from 1 to 3 substituents independently selected from the group consisting of C₁₋₃ alkyl, halogen, C₁₋₃ alkoxy, CO₂C₁₋₆ alkyl, C₁₋₆ thioalkyl, OH, cyano, nitro, N(C₁₋₃ alkyl)₂ and phenyl, where the phenyl is optionally substituted with from 1 to 3 substituents independently selected from the group consisting of C₁₋₃ alkyl, halogen, C₁₋₃ alkoxy, CO₂C₁₋₆ alkyl, C₁₋₆thioalkyl, OH, cyano, nitro, N(C₁₋₃ alkyl)₂ and phenyl; or a pharmaceutically acceptable salt thereof.

In a preferred embodiment, the invention provides the compound (16β,17a)-3,16-bis(benzyloxy)estra-1,3,5(10)-trien-17-ol, or a pharmaceutically acceptable salt thereof.

### DETAILED DESCRIPTION OF THE INVENTION

The present invention provides new and useful methods for the production of important d-ring substituted steroids. Specifically, this invention provides for an epoxide ring-opening reaction in a steroid d-ring that generates an otherwise very difficult to obtain, 16β-alkoxy, 17α-hydroxy steroid that can be further converted to steroidal 16β,17α-diols. Steroids possessing a 3-hydroxy estratriene nucleus and a 16,17-diol are commonly known as estriols and have important estrogenic biological activity with found utility in hormone replacement regimens. The estriol, 1,3,5(10)-estratriene-3,16β,17α-triol, is a component of the important estrogen replacement therapy, Premarin®. In the case of the Premarin® products, the phenolic hydroxyl group may be a free phenol or alternatively, conjugated via a sulfate.

The present invention provides, inter alia, general and novel processes for the preparation of 16β-alkoxy, 17α-hydroxy substituted steroids comprising the reaction of an α-epoxy moiety with an alcoholate according to the process described in scheme I:

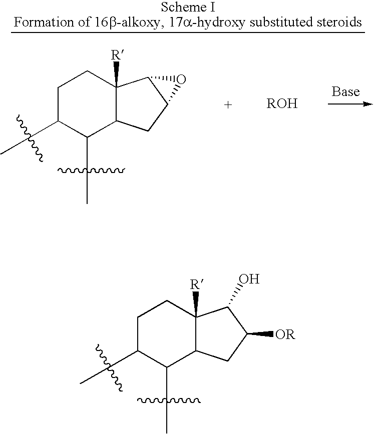

The present invention further provides for the preparation of 16β,17α-steroidal diols via the overall transformation illustrated in Scheme II, wherein the α-epoxide is opened and then the ether deprotected to form the steroidal c, d-ring diol.

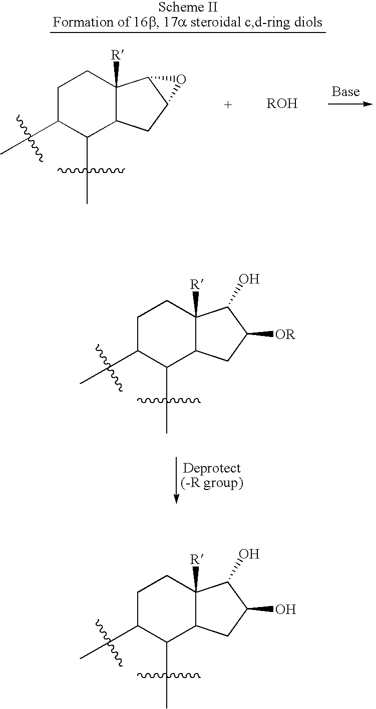

In some embodiments, the alcohol used to open the α-epoxy steroid is a benzyl alcohol, which allows for convenient subsequent deprotection and concomitant generation of the steroidal 16β,17α-diol (Scheme III).

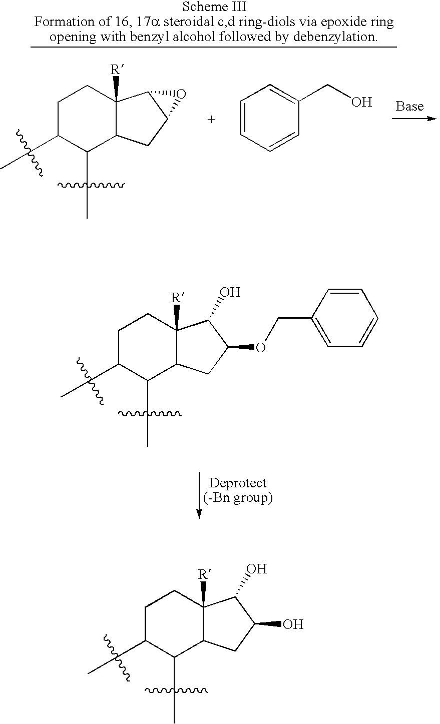

The ring opening epoxide reaction is a useful method for making 16β-alkoxy, 17α-hydroxy steroids of many types. For example, it is useful for gonanes, estranes and androstanes. In some embodiments, 1,3,5(10)-estratrienes serve as the core steroidal skeleton, as illustrated in Scheme IV.

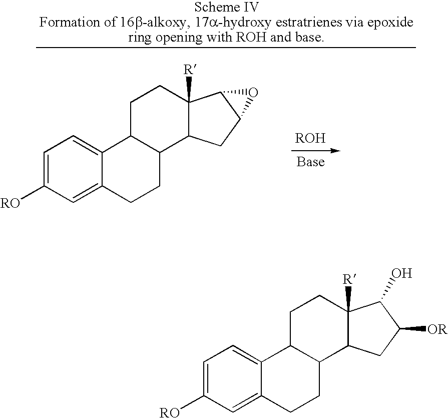

The 16β-alkoxy, 17α-alkoxy estratriene illustrated in scheme IV can be further elaborated to a 1,3,5(10)-estratriene-3,16β,17α-triol. If the 3-phenolic R group is other than hydrogen, the deprotection sequence may rely on either sequential or simultaneous deprotection of the 3-OR and 16-OR group. The epoxide ring-opening reaction and subsequent deprotection reactions just described are illustrated in Scheme V.

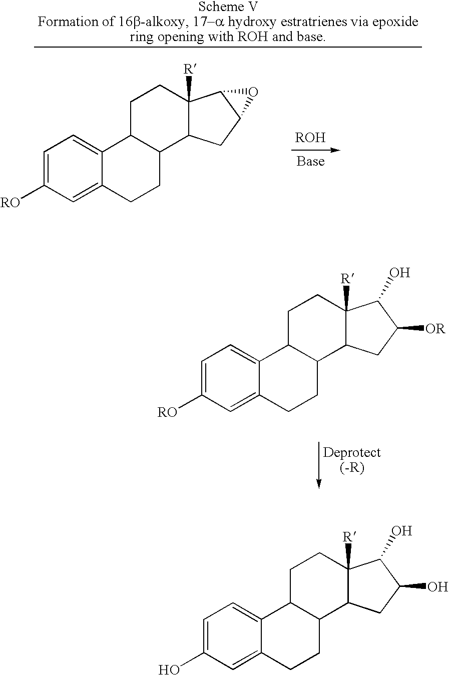

The steroidal d-ring epoxides used as starting materials in Schemes I-V can be prepared using a variety of methods, including for example, stereoselective epoxidation of a double bond. The base used in the ring opening epoxide reaction is, preferably a base that is strong enough to significantly deprotonate the alcohol nucleophile ROH. Such bases include, but are not limited to, metal alkoxides such as sodium ethoxide, potassium ethoxide, sodium t-butoxide, potassium t-butoxide, and the like. Alkyl metals such as alkyl or aryl lithiums and alkyl or aryl magnesiums also may be used. Strong bases consisting of metal salts of amines also may be used, e.g., lithium diisopropylamide (LDA). Group I or II metal hydride salts such as lithium hydride, sodium hydride, potassium hydride, calcium hydride and the like also are useful.

The reaction can be performed by first substantially deprotonating the alcohol and then adding the steroidal epoxide to the alkoxide thus formed. In some embodiments, the alcohol can be deprotonated and then added to the steroidal epoxide. In yet other embodiments, the alcohol, base and steroidal epoxide are combined together and then treated with the appropriate base.

In some embodiments, the deprotonation of the alcohol can be assisted by heating of the alcohol and base. In some embodiments, the alcohol and base are heated to greater than about 40° C., and in some embodiments, between about 50° C. and about 60° C. In some embodiments, the base and alcohol are heated for a time between about 1 minute and about 120 minutes, or between about 5 minutes and about 60 minutes, or about 15 minutes. In some embodiments, the alcohol is benzyl alcohol and the base is sodium t-butoxide.

In some embodiments, it may be preferable that the alcohol is deprotonated in a solvent that is different than the alcohol. In some embodiments, it is advantageous to deprotonate the alcohol using the same alcohol as the reaction solvent. In some such embodiments, the alcohol nucleophile is benzyl alcohol and benzyl alcohol also is the solvent. In some embodiments, the benzyl alcohol is used in greater than about 1 equivalent, in some embodiments greater than about 5 equivalents, and in some embodiments greater than about 10 equivalents.

In some embodiments, a co-solvent can be added to the reaction to increase reaction yield and suppress undesired by-product formation. In some embodiments, such co-solvents can be such solvents such as DMSO, HMPA, HMPT, 1-methyl-2-pyrrolidinone, DMF, dimethyl acetamide, and the like. In some embodiments, 1-methyl-2-pyrrolidinone is added together with the epoxysteroid to the alcohol and base. In some embodiments, from between about 0.1 and about 2.0 equivalents (equivalents based on epoxysteroid) of 1-methyl-2-pyrrolidinone are added, or between about 0.5 and about 1.0 equivalents.

Depending on the reactivity of the particular alcohol nucleophile and steroidal epoxide reactant, it may be advantageous to heat the reaction for an appropriate time and at an appropriate temperature to maximize product yield. In some embodiments, it might be preferred to heat the reaction at various temperatures for various periods of time in order to maximize product yield. In some embodiments, the reaction mixture is heated at between about 50° C. and about 200° C., in some embodiments the reaction mixture is heated at between about 100° C. and about 160° C., in some embodiments between about 130° C. and about 150° C. In some embodiments, the reaction is heated for between about 1 hour and about 60 hours, in some embodiments, between about 5 hours and about 40 hours, in some embodiments, between about 10 hours and about 30 hours. In some embodiments, the reaction is heated at about 140° C. for about 21 hours and then at about 145° C. for about 7 hours.

The product of the reaction may be isolated by various techniques known in the art. For example, in some embodiments, it might be preferable to isolate the reaction product by quenching with an appropriate acid followed by extraction and chromatography. Alternatively, it might be preferable in some embodiments to quench the reaction and directly precipitate the product by addition of a suitable solvent. In some such embodiments, the solvent can be an alkyl alcohol such as iso-propyl alcohol, ethanol or methanol. In some embodiments, the product can be precipitated out by the addition of an alcohol followed by the addition of water. Where the reacting alcohol is used as the reaction solvent, the reaction mixture can be treated with a volume of an alcohol followed by a volume of water, and the product isolated. In some embodiments, the volume of solvent alcohol, to alcohol added, to water added is in a ratio of from about 0.2 to 0.6 to about 0.5 to 2 to about 0.4 to 1.2, respectively. In some embodiments, the volume of solvent alcohol, to alcohol added, to water added is in a ratio of from 0.4 to 1 to 0.8, respectively. In some embodiments, the reactant alcohol and solvent is benzyl alcohol and the alcohol added is methanol.

The isolated product may be further purified by washing with an appropriate solvent, or mixture of solvents, or may be washed multiple times by a solvent or mixture of solvents. Well-suited solvents or mixtures of solvents are better at dissolving impurities in the product than dissolving the product itself. In some embodiments, the product is washed with an alcohol, or with water, or with a mixture of alcohol and water. One suitable alcohol for the wash is methanol. In some embodiments, the methanol to water ratio is respectively, 1:4, v/v. Yields of greater than about 50%, about 60%, about 70%, about 80%, about 90% or about 97% can be obtained in this manner, and purity greater than about 80%, about 85%, about 90%, about 95%, or about 99% can be obtained without further purification steps.

In some embodiments, the product can be further purified by recrystallization. The recrystallization can be performed with a solvent or with a mixture of solvents. Suitable solvents for the mixture include hydrocarbon solvents together with alcohol solvents. Examples of suitable hydrocarbon solvents include pentane, hexane or heptane, or mixtures thereof. Suitable alcohols include alkyl alcohols, for example, and without limitation, methanol, ethanol, propyl alcohol, iso-propyl alcohol, butanol or 2-butyl alcohol. In some embodiments, the recrystallization solvent is a mixture of iso-propyl alcohol and heptane. In some embodiments, the recrystallization solvent mixture of iso-propyl alcohol and heptane is used in a ratio of 1:8, v/v.

Schemes II, III and V describe processes for the production of steroidal d-ring diols. For these processes, the removal of the R-group(s) can be performed by standard deprotection procedures well-known to those of skill in the art. In embodiments where R is a benzyl group, numerous reductive and non-reductive methods can be utilized for the removal of this group. Non-reductive methods include HI, HBr, TMSI and the like. Reductive procedures include procedures utilizing hydrogen either directly via the employment of hydrogen gas and a catalyst or via hydrogen transfer using a moiety such as cyclohexadiene and a catalyst. In some embodiments, the metal used is Pd on carbon. In some embodiments, the Pd is present in an amount from 5% to 10% by weight. For a general description of some deprotection procedures including the deprotection of alkyl and benzyl ethers, see “Protective Groups in Organic Synthesis” by Greene and Wuts, 3^{rd} edition, 1999, John Wiley and Sons, which is hereby incorporated by reference.

As used herein, the term “alkyl” or “alkylene” is meant to refer to a saturated hydrocarbon group, which is straight-chained or branched. Example alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl) and the like. An alkyl group can contain from 1 to about 20, from 2 to about 20, from 1 to about 10, from 1 to about 8, from 1 to about 6, from 1 to about 4, or from 1 to about 3 carbon atoms.

As used herein, “alkenyl” refers to an alkyl group having one or more double carbon-carbon bonds. Example alkenyl groups include ethenyl, propenyl, butenyl, pentenyl, hexenyl, butadienyl, pentadienyl, hexadienyl, and the like.

As used herein, “alkynyl” refers to an alkyl group having one or more triple carbon-carbon bonds. Example alkynyl groups include ethynyl, propynyl, butynyl, pentynyl, and the like.

As used herein, “halo” or “halogen” refers to fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

The term “steroid” is intended to have its accustomed meaning as compounds that are derivatives of the perhydrocyclopentanophenanthrene, having the general ring structure: 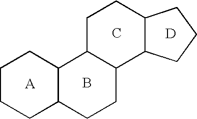
where the letters A-D indicate the respective rings of the steroid.

As used herein, the term “reacting” refers to the bringing together of designated chemical reactants such that a chemical transformation takes place, generating a compound different from any initially introduced into the system. Reacting can take place in the presence or absence of solvent.

At various places in the present specification substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl and C₆ alkyl.

The compounds of the present invention can contain an asymmetric atom, and some of the compounds can contain one or more asymmetric atoms or centers, which thus, can give rise to optical isomers (enantiomers) and diastereomers. The present invention includes such optical isomers (enantiomers) and diastereomers (geometric isomers), as well as the racemic and resolved, enantiomerically pure R and S stereoisomers, and other mixtures of the R and S stereoisomers and pharmaceutically acceptable salts thereof. Optical isomers can be obtained in pure form by standard procedures known to those skilled in the art, and include, but are not limited to, diastereomeric salt formation, kinetic resolution, and asymmetric synthesis. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

The processes described herein can be monitored according to any suitable method known in the art. For example, product formation can be monitored by spectroscopic means such as nuclear magnetic resonance spectroscopy (e.g., ¹H or ¹³C), infrared spectroscopy, spectrophotometry (e.g., UV-visible), and mass spectrometry, or by chromatography such as high performance liquid chromatograpy (HPLC) and thin layer chromatography.

The reactions of the processes described herein can be carried out in suitable solvents, which can be readily selected by one of skill in the art of organic synthesis. Suitable solvents can be substantially nonreactive with the starting materials (reactants), the intermediates, or products at the temperatures at which the reactions are carried out, e.g., temperatures that can range from the solvent's freezing temperature to the solvent's boiling temperature. Thus, a wide variety of solvents are amenable to the present invention. In some preferred embodiments, the solvent is an alcohol of formula HO—R, as described herein. One preferred such solvent is benzyl alcohol.

The reactions of the processes described herein can be carried out at appropriate temperatures, which can be readily determined by the skilled artisan. Reaction temperatures will depend on, for example, the melting and boiling points of the reagents and solvent, if present, the thermodynamics of the reaction (e.g., vigorously exothermic reactions typically are carried out at reduced temperatures), and the kinetics of the reaction (e.g., a high activation energy barrier typically necessitates elevated temperatures). “Elevated temperatures” refers to temperature above room temperature (about 20° C.) and “reduced temperatures” refers to temperatures below room temperature.

The reactions of the processes described herein can be carried out in air or under an inert atmosphere. Typically, reactions containing reagents or products that are substantially reactive with air can be carried out using air-sensitive synthetic techniques that are well known to the skilled artisan.

It is appreciated that certain features of the invention, which are, for clarity, described in the context of separate embodiments, also can be provided in combination in a single embodiment. Conversely, various features of the invention, which are, for brevity, described in the context of a single embodiment, also can be provided separately or in any suitable subcombination.

The processes of this invention are suitable for the preparation of compounds of Formula I on any convenient scale, for example, greater than about 0.01 mg, 0.10 mg, 1 mg, 10 mg, 100 mg, 1 g, 10 g, 100 g, 1 kg, 10 kg or more. The processes are particularly advantageous for the large scale (e.g., greater than about ten grams) preparation of 16β-alkoxy, 17α-hydroxy steroids and steroidal 16β,17α-diols.

The invention will be described in greater detail by way of specific examples. The following examples are offered for illustrative purposes, and are not intended to limit the invention in any manner. Those of skill in the art readily will recognize a variety of noncritical parameters, which can be changed or modified to yield essentially the same results.

### EXAMPLES

### Example 1

### PREPARATION OF (16β,17α)-3,16-BIS(BENZYLOXY)ESTRA-1,3,5(10)-TRIEN-17-OL

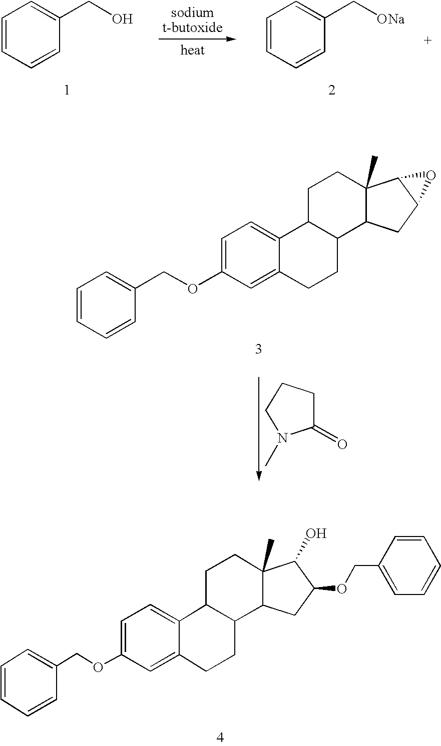

To benzyl alcohol (1, 600 g, 5.55 mol) was added sodium t-butoxide (40.0 g, 0.416 mol) in portions. The mixture was heated to 55° C. and stirred for 15 min. Then the epoxide (reg no 35565-68-5) (3, 200 g, 0.555 mol) and 1-methyl-2-pyrrolidinone (40 g, 0.40 mol) were added. The mixture was heated to 140° C. and stirred for 21 h and 145° C. for 7 h. The mixture was cooled to room temperature and methanol (1500 mL) was added. Then water (1200 mL) was added. The solid was filtered and washed with a mixture of methanol and water (1:4, v/v). The wet solid was dried to give a crude product (235 g) that was recrystallized from iso-propyl alcohol and heptane (1:8, v/v) to yield a white solid (4, 210 g, 79%). ¹H NMR (CDCl₃): δ (10H, m), 7.2 (1H, d, j=8.5 Hz), 6.78 (1H, dd, j=2.7, 8.5 Hz), 6.72 (1H, d, j=2.7 Hz), 5.03 (2H, s), 4.57 (2H, s), 3.82 (2H, m), 2.87 (2H, m), 2.18-2.42 (3H, m), 1.9 (1H, m), 1.36-1.79. (8H, m), 0.92 (3H, s).

### Example 2

### PREPARATION OF 1,3,5(10)-ESTRATRIENE-3,16B,17A-TRIOL

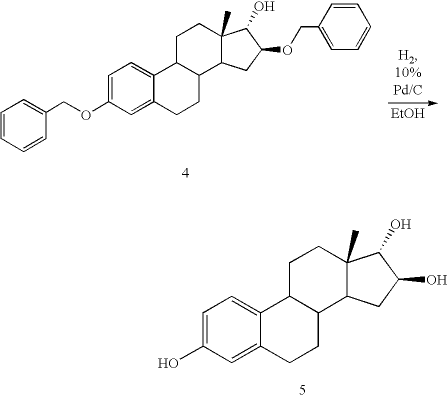

Compound 4 is dissolved in EtOH and treated with a catalytic amount of 10% Pd/C and an atmosphere of hydrogen gas is applied. The reaction is monitored periodically by HPLC for completion. When the reaction is complete, the hydrogen atmosphere is exchanged with nitrogen and the reaction mixture is filtered through a bed of Celite® to remove the catalyst. The solvent is evaporated to provide estriol 5.

As those skilled in the art will appreciate, numerous changes and modifications may be made to the preferred embodiments of the invention without departing from the spirit of the invention. It is intended that all such variations fall within the scope of the invention. It is intended that each of the patents, applications, and printed publications, including books, mentioned in this patent document be hereby incorporated by reference in their entirety.

What is claimed is:

1. A process for preparing a steroid having the c, d-ring structure of Formula I: 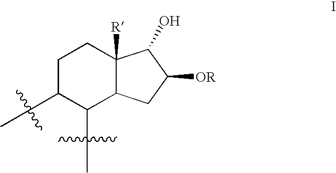
wherein:
R is C₁₋₆ alkyl or benzyl wherein the phenyl ring of the benzyl group is optionally substituted with from 1 to 3 substituents independently selected from the group consisting of C₁₋₃ alkyl, halogen, C₁₋₃ alkoxy, CO₂C₁₋₆ alkyl, C₁₋₆ thioalkyl, OH, cyano, nitro, N(C₁₋₃ alkyl)₂ and phenyl; and
R′ is C₁₋₃ alkyl; comprising:
a) reacting a steroid having the c, d-ring structure of Formula II: 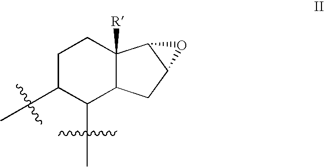
with a compound of formula HO—R in the presence of a base for a time and under conditions effective to form the compound of Formula I.

2. The process of claim 1 wherein R is benzyl and R′ is methyl.

3. The process of claim 1 wherein the base is a group I or II metal hydride or metal t-butoxide.

4. The process of claim 1 wherein the base is sodium t-butoxide.

5. The process of claim 1 wherein the reaction is performed in a solvent.

6. The process of clam 5 wherein the solvent is benzyl alcohol.

7. The process of claim 1 wherein the reaction of step (a) further comprises a cosolvent.

8. The process of claim 7 wherein the cosolvent is 1-methyl-2-pyrrolidinone.

9. The process of claim 8 wherein the reaction of step (a) comprises adding the steroid having the c, d-ring structure of Formula II and the 1-methyl-2-pyrrolidinone to a mixture of the compound of Formula R—OH and the base.

10. The process of claim 1 wherein R is benzyl; R′ is methyl; the base is sodium t-butoxide; and the reaction of step (a) is performed in excess benzyl alcohol solvent, with a cosolvent that is 1-methyl-2-pyrrolidinone.

11. The process of claim 10 wherein the reaction of step (a) comprises:
(i) reacting the sodium t-butoxide with an excess of the benzyl alcohol to form a reaction mixture thereof; and
(ii) adding a mixture of the compound of Formula II and the 1-methyl-2-pyrrolidinone to the reaction mixture of step (i).

12. The process of claim 1 further comprising the step of:
(b) removing the group R from the steroid having the c, d-ring structure of Formula I to provide a steroid having the c, d-ring structure of Formula III:

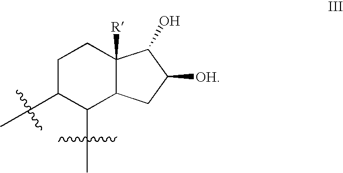

13. The process of claim 11 further comprising the step of:
(b) removing the group R from the steroid having the c, d-ring structure of Formula I to provide a steroid having the c, d - ring structure of Formula III:

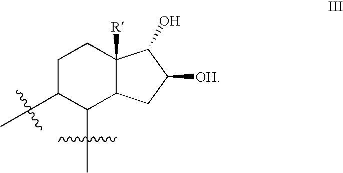

14. The process of claim 12 wherein the removing of step (b) is performed with hydrogen and a metal catalyst.

15. The process of claim 13 wherein the removing of step (b) is performed with hydrogen and a metal catalyst.

16. The process of claim 14 wherein the metal catalyst is Pd on carbon.

17. The process of claim 15 wherein the metal catalyst is Pd on carbon.

18. The process of claim 11 wherein the steroid having the c, d-ring structure of Formula II has the structure: 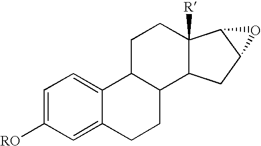
and the steroid having the c, d-ring structure of Formula I has the structure: 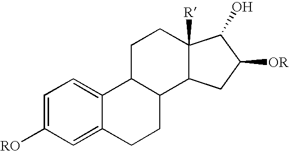
wherein each R is benzyl; and R′ is methyl.

19. The process of claim 15 wherein the steroid having the c, d-ring structure of Formula II has the structure: 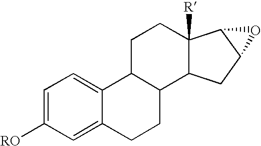
the steroid having the c, d-ring structure of Formula I has the structure: 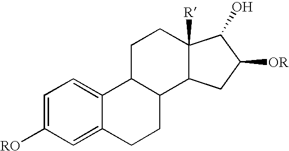
wherein each R is benzyl; and R′ is methyl; and the steroid having the c, d-ring structure of Formula III has the structure:

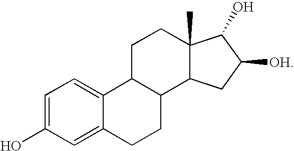

20. The process of claim 11 wherein the benzyl alcohol is heated to greater than about 40° C. after being reacted with sodium t-butoxide.

21. The process of claim 11 wherein the benzyl alcohol is heated to between about 50° C. and about 60° C. after being reacted with sodium t-butoxide.

22. The process of claim 21 wherein the benzyl alcohol is heated for between 5 minutes and 60 minutes after the addition of the sodium t-butoxide.

23. The process of claim 11 wherein the reaction mixture is heated after the addition of the steroid having the c, d-ring structure of Formula II.

24. The process of claim 11 wherein the reaction mixture is heated to greater than about 100° C. after the addition of the steroid having the c, d-ring structure of Formula II.

25. The process of claim 11 wherein the reaction mixture is heated to between about 130° C. and about 150° C. after the addition of the steroid having the c, d-ring structure of Formula II.

26. The process of claim 25 wherein the reaction mixture is heated from about 5 to about 40 hours.

27. The process of claim 26 wherein the reaction mixture is heated at about 140° C. for about 21 hours and at about 145° C. for about 7 hours.

28. The process of claim 27 wherein the product having the c, d-ring structure of Formula I is collected by precipitation.

29. The process of claim 27 wherein the precipitation is effected by cooling the reaction mixture and adding methanol and water.

30. The process of claim 29 wherein the ratio of the initial volume of benzyl alcohol, to the volume of methanol added, to the volume of water added, is respectively, about 0.4 to about 1 to about 0.8.

31. The process of claim 28 wherein the precipitated product having the c, d-ring structure of Formula I is isolated and washed with a mixture of an alcohol and water.

32. The process of claim 31 wherein the alcohol is methanol.

33. The process of claim 32 wherein the methanol to water ratio in the wash is about 1 volume to about 4 volumes, respectively.

34. The process according to claim 16 wherein the Pd is present in an amount from 5 to 10% by weight.

35. The process according to claim 17 wherein the Pd is present in from 5 to 10% by weight.

36. A compound having the formula V: 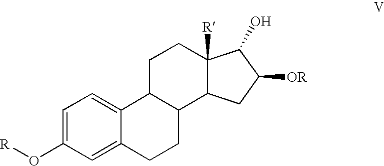
wherein:
R′ is C₁₋₃ alkyl; and
each R is an independently selected benzyl group wherein the phenyl ring of each benzyl group is optionally substituted with from 1 to 3 substituents independently selected from the group consisting of C₁₋₃ alkyl, halogen, C₁₋₃ alkoxy, CO₂C₁₋₆ alkyl, C₁₋₆ thioalkyl, OH, cyano, nitro, N(C₁₋₃ alkyl)₂ and phenyl; wherein the phenyl is optionally substituted with from 1 to 3 substituents independently selected from the group consisting of C₁₋₃ alkyl, halogen, C₁₋₃ alkoxy, CO₂C₁₋₆ alkyl, C₁₋₆ thioalkyl, OH, cyano, nitro, N(C₁₋₃ alkyl)₂ and phenyl;
or a pharmaceutically acceptable salt thereof.

37. The compound of claim 36 that is (16β,17α)-3,16-bis(benzyloxy)estra-1,3,5(10)-trien-17-ol, or a pharmaceutically acceptable salt thereof.

38. A product of the process of any of claims 1-35.
